(19) United States
(12) Patent Application Publication
(10) Pub. No.: US 2022/0395656 A1
(43) Pub. Date: Dec. 15, 2022

(54) PRINTED ELECTRONICS BREATH INDICATOR

(71) Applicant: Vyaire Medical, Inc., Mettawa, IL (US)

(72) Inventor: Dennis R. WHITE, Yorba Linda, CA (US)

(21) Appl. No.: 17/764,129

(22) PCT Filed: Sep. 25, 2020
(86) PCT No.: PCT/US2020/052873

§ 371 Date: Mar. 25, 2022

Related U.S. Application Data
(60) Provisional application No. 62/907,492, filed on Sep. 27, 2019.

(51) Int. Cl. A61M 16/06; A61M 16/00

(52) U.S. Cl. CPC A61M 16/06; A61M 16/0003; A61M 2205/0227; A61M 2230/432; A61M 2230/435; A61B 2562/166

(57) ABSTRACT

The disclosed systems and methods provide a printed electronics breath indicator that may be directly printed or molded onto a manufactured breathing device, or separately attached to an existing breathing device. The printed electronics breath indicator may include an electroluminescent indicator, a sensor, and a processor. The processor may be configured to receive sensor data from the sensor, the sensor data including detected carbon dioxide (CO2) or oxygen (O2) levels. The processor may be further configured to determine a current breathing state of the patient based on the received sensor data. The processor may be further configured to cause the electroluminescent indicator to generate a visual representation according to the determined current breathing state of the patient. The visual representation may comprise a variable visual representation that displays one or more visual parameters that are proportional to the detected CO2 or O2 levels.

### BACKGROUND FIELD

The subject technology addresses deficiencies in indicating patient breathing state, as commonly encountered in hospital care settings.

### SUMMARY

In hospitals or other patient care settings, an easily identifiable indication of patient breathing state enables caregivers to better monitor patient health and improve patient outcomes. For example, anesthesiologists need to confirm that a patient is breathing normally after medicine is administered. One approach to identifying patient breath may be to observe condensation that forms on a mask or other breathing device of a patient. However, this condensation may not always be easily visible, and may not be applicable where breathing devices lack a suitable translucent surface to view condensation. As described herein, a custom designed indicator device may be attached to breathing devices, and may employ chemically reactive materials that visibly change color according to carbon dioxide (CO2) levels in the patient's breath.

Conventional chemically reactive color changing materials may suffer from a number of drawbacks. For example, since the chemically reactive materials may be sensitive to atmospheric exposure, costly specialized packaging may be required to preserve the efficacy of the materials prior to use. Once opened and in use, the materials may become saturated with CO2 over time, rendering color changes, and in turn actual CO2 levels, more difficult to discern. Further, the materials are often opaque, which may obscure portions of the patient that should be visually unobstructed for ideal patient care. Yet further, it may be desirable to monitor and perform analytics on various characteristics of the patient's breath, which may not be possible with conventional indicator materials. Accordingly, there is a need for an improved breath indicator device which can remedy these deficiencies.

According to various implementations, a method for providing an indication of breath using a printed electronics breath indicator is provided. The method may include receiving sensor data from a sensor, the sensor data including detected carbon dioxide (CO2) or oxygen (O2) levels. The sensor data may also include temperature, humidity, breath biomarkers, and other data. The method may further include determining a current breathing state of a patient based on the received sensor data. The method may further include causing an electroluminescent indicator to generate a variable visual representation according to the determined current breathing state of the patient.

Other aspects include corresponding systems, apparatuses, and computer program products for implementation of the computer-implemented method.

Further aspects of the subject technology, features, and advantages, as well as the structure and operation of various aspects of the subject technology are described in detail below with reference to accompanying drawings.

### DESCRIPTION OF THE FIGURES

Various objects, features, and advantages of the present disclosure can be more fully appreciated with reference to the following detailed description when considered in connection with the following drawings, in which like reference numerals identify like elements. The following drawings are for the purpose of illustration only and are not intended to be limiting of this disclosure, the scope of which is set forth in the claims that follow.

FIG. 1 depicts an example system for using a printed electronics breath indicator, according to various aspects of the subject technology.

FIG. 2A and FIG. 2B depict an example mask using a printed electronics breath indicator to indicate CO2 levels during patient inhalation and exhalation, according to various aspects of the subject technology.

FIG. 2C depicts alternative breathing devices using a printed electronics breath indicator to indicate CO2 levels during patient inhalation and exhalation, according to various aspects of the subject technology.

FIG. 2D depicts a perspective view of an open mask using a printed electronics breath indicator to indicate CO2 levels during patient inhalation and exhalation, according to various aspects of the subject technology.

FIG. 2E depicts a rear view of the open mask of FIG. 2D, according to various aspects of the subject technology.

FIG. 3A and FIG. 3B depict a close-up view of example components of a printed electronics breath indicator during patient inhalation and exhalation, according to various aspects of the subject technology.

FIG. 4 depicts an example process for a printed electronics breath indicator providing an indication of breath, according to various aspects of the subject technology.

FIG. 5 is a conceptual diagram illustrating an example electronic system for providing a printed electronics breath indicator, according to various aspects of the subject technology.

### DETAILED DESCRIPTION

While aspects of the subject technology are described herein with reference to illustrative examples for particular applications, it should be understood that the subject technology is not limited to those particular applications. Those skilled in the art with access to the teachings provided herein will recognize additional modifications, applications, and aspects within the scope thereof and additional fields in which the subject technology would be of significant utility.

The subject technology provides a flexible, translucent breath indicator circuit that includes one or more sensors and corresponding electronics directly printed or molded, etched, or otherwise fused onto a substrate of the circuit and configured to detect a molecular component portion of a patient's breath, such as CO2 or oxygen, or a physical state, such as temperature, pressure, or pH level. A substrate of the breath indicator circuit may be part of a manufactured breathing device, or a separate material component onto which the circuit components are fused, and subsequently attached to an existing breathing device or device component. By using a controller or another computing device, data from the sensor(s) can be intelligently interpreted in a consistent and reliable manner. Once the signals are electronically interpreted by the controller, the controller may change a color, intensity, or other parameters of an indicator, such as a flexible organic light-emitting diode (OLED) array associated with the device by way of, for example, being printed or molded onto the substrate. Further, since the sensor data is gathered by a controller, the controller may additionally transmit the sensor data to a remote data store for storage, analysis and/or further distribution. Transparent materials may be used for the indicator, the sensor(s), conductors and/or the OLED array, to provide a clear and unobstructed view of the patient. Since the indicator may be manufactured to be resilient against atmospheric exposure, manufacturing costs can be reduced by using conventional materials thereby rendering the indicator more suitable for use with disposable breathing devices.

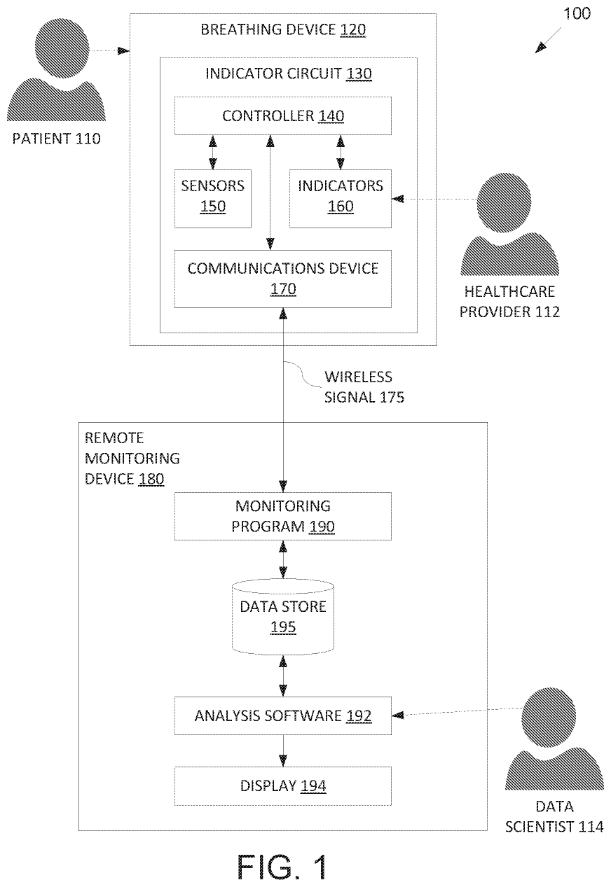

FIG. 1 depicts an example system 100 for using a printed electronics breath indicator circuit 130, including a breathing device 120, according to various aspects of the subject technology. Breathing device 120 includes indicator circuit 130. Indicator circuit 130 includes controller 140, sensors 150, indicators 160, and communications device 170. Remote monitoring device 180 includes monitoring program 190, analysis software 192, and data store 195.

As shown in system 100, breathing device 120 may be fitted on a patient 110. Breathing device 120 may be, for example, a mask, a nasal cannula, a resuscitation device, or any other breathing device, and may also be a disposable device. A healthcare provider 122, which may correspond to a healthcare professional such as a doctor or nurse, may require an easily recognizable indication of the breathing state of patient 110, which indicator circuit 130 may provide. Indicator circuit 130 may be manufactured as part of breathing device 120 or separately attached to breathing device 120, allowing indicator circuit 130 to be used with existing breathing devices 120 that do not have built-in breath indicator features.

Indicator circuit 130 may include several components, as shown in system 100. Indicator circuit 130 may be a flexible printed circuit, allowing indicator circuit 130 to conform to curved, irregular, and/or flexible non-rigid surfaces of breathing device 120. For example, indicator circuit 130 may be fabricated using one or more electronics printing techniques such as, for example, inkjet printing, screen printing, aerosol jet printing, evaporation printing, or other methods. Indicator circuit 130 may be, for example, directly printed onto one or more substrate surfaces of breathing device 120, or attached as part of a molded element in a plastic injection process. Alternatively, indicator circuit 130 may be, for example, printed onto a separate substrate that can be coupled to breathing device 120 using one or more attachment devices, such as, for example, adhesives, connectors, fasteners, magnets, clasps, straps, buckles, or other similar devices. The substrates may comprise, for example, transparent or translucent, flexible, elastic and stretchable thin films or multi-layered films using materials such as, for example, polyethylene terephthalate (PET), polyethylene naphthalate (PEN), or polyimide (PI) films or layers. It is envisioned that other polymers, copolymers and composite type materials and structures can be used in manufacturing the indicator circuit 130. Conductive traces and/or conductive inks printed on the substrates may provide electrical connections between the components of indicator circuit 130. To minimize visual obstruction of patient 110, the circuitry of indicator circuit 130 may use small and efficient micro-sized components and highly integrated circuit packages to reduce the footprint of indicator circuit 130. In this manner, indicator circuit 130 can adapt to a breathing device 120 of various shapes, sizes, materials and rigidity.

Controller 140 may correspond to any type of general or specialized processor, controller, integrated circuit, application specific integrated circuit (ASIC), field programmable gate array (FPGA), system-on-chip, or similar device, and may include hardcoded circuit elements, firmware, software, or any combination thereof. Controller 140 may receive sensor data from sensors 150, which may include CO2 sensors. Based on the received sensor data, controller 140 may modify indicators 160 to provide an easily visible indication to healthcare provider 112 of the current breathing state of patient 110. In some implementations, the visible indication may include non-visible light, for example infrared or ultraviolet light waves, which can be perceived using night vision equipment or other devices. Indicators 160 may include any type of electroluminescent indicator such as an OLED array, a phosphorescent display, a luminescent element, a LED array, or another device that is drivable by controller 140.

Further, since controller 140 is already receiving sensor data from sensors 150, controller 140 may optionally utilize communications device 170 to transfer the sensor data to remote monitoring device 180 via wireless signal 175. For example, communications device 170 may include a transceiver that transmits and receives wireless signals 175 by way of radio waves, Bluetooth or Bluetooth Low Energy, Wi-Fi, or any other wireless protocol to transmit data. While wired transmission is also possible, it may be undesirable to have extra wires connected to breathing device 120. Further, while a network is not specifically illustrated in system 100, it should be understood that an infrastructure or ad-hoc network with one or more intervening nodes may be present between breathing device 120 and remote monitoring device 180.

While a power source is not specifically illustrated in system 100, it should be understood that indicator circuit 130 may be coupled to a power source or, in some implementations, receive power wirelessly from a power source. For example, the power source may include one or more of a rechargeable battery, a non-rechargeable battery, a capacitor, a wireless power harvester, a solar panel, an inductive charging coil, or any other combination of storage and harvesting elements. Further, communications device 170 may comprise portions or the entirety of the power source. For example, electrical energy may be obtained from radio frequency identification (RFID) technology by way of inductive coupling to an external power source. In some embodiments, communications device 170 may include a coil or coupling capacitor for power harvesting only. In some embodiments, communications device 170 may be omitted entirely, enabling indicator circuit 130 to provide a cost reduced and standalone breath indicator solution.

In implementations in which communications device 170 is present and includes wireless radios for transmission, monitoring program 190 may be executing on remote monitoring device 180 to receive wireless signal 175 from communications device 170. Remote monitoring device 180 may be a smartphone, tablet, laptop, desktop, or other computing device. Monitoring program 190 may collect and aggregate the sensor data collected from sensors 150 on a per-patient basis, and may store the sensor data in data store 195. While data store 195 is depicted as being part of monitoring device 180, data store 195 may be located on a remote server or another device. Remote monitoring device 180 and indicator circuit 130 may be configured to comply with all relevant regulatory laws regarding the storage and privacy of electronic health records, such as the Health Insurance Portability and Accountability Act (HIPAA). For example, health records may be protected with strong encryption to prevent third party access, and further be erased from non-volatile memory when no longer necessary for retention.

Monitoring program 190 may also generate alerts and notifications based on the sensor data, for example if the sensor data satisfies a low or high threshold for a particular sensor reading, such as detected CO2 levels at an instant in time or over a period of time. These alerts and notifications may generate a text message, a visual stimulus, an audible alarm, or another type of alert directed to a healthcare provider, such as healthcare provider 112, or to other medical professionals.

Further, analysis software 192 may be provided to allow a user, such as a data scientist 114, to perform analytics on the collected sensor data, which may occur concurrently with data collection or after sufficient data history is recorded in data store 195. For example, the analytics may be utilized to identify health trends, to determine cause and correlation of various health parameters, and to provide advanced warning of potential health issues for preventative care. While analysis software 192 is shown as part of remote monitoring device 180, analysis software 192 may instead be provided on a separate device or workstation, or incorporated into software/firmware located on indicator circuit 130. Further, depending on the access privileges of data scientist 114 and local regulatory requirements, analysis software 192 may only allow access to data store 195 on an aggregated basis, thereby preventing access to individual patient data.

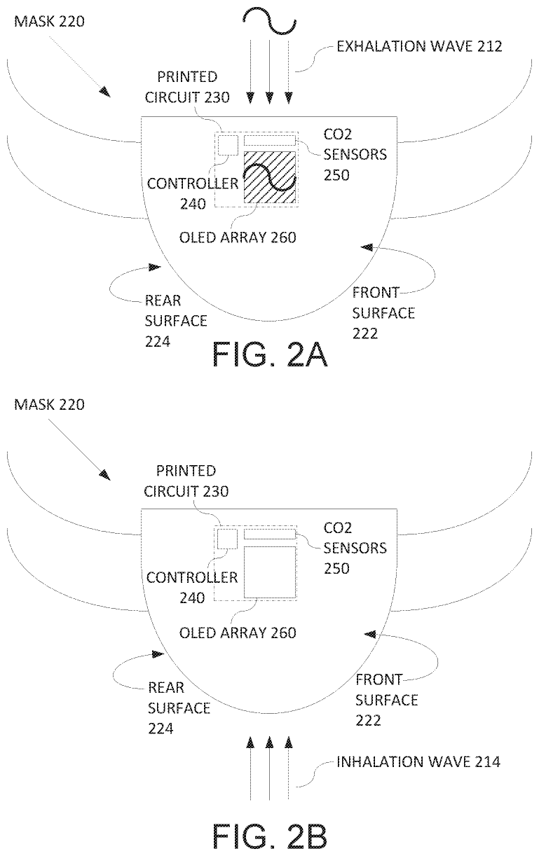

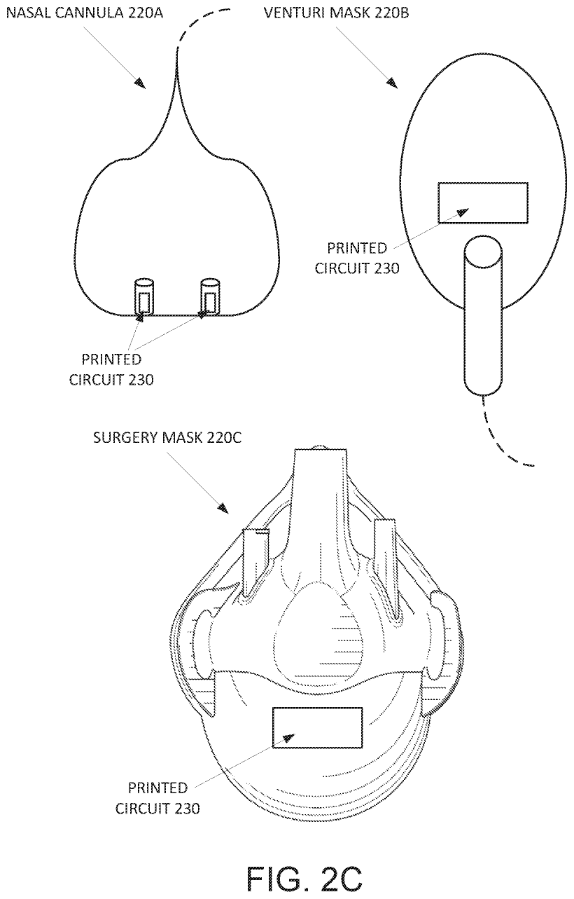

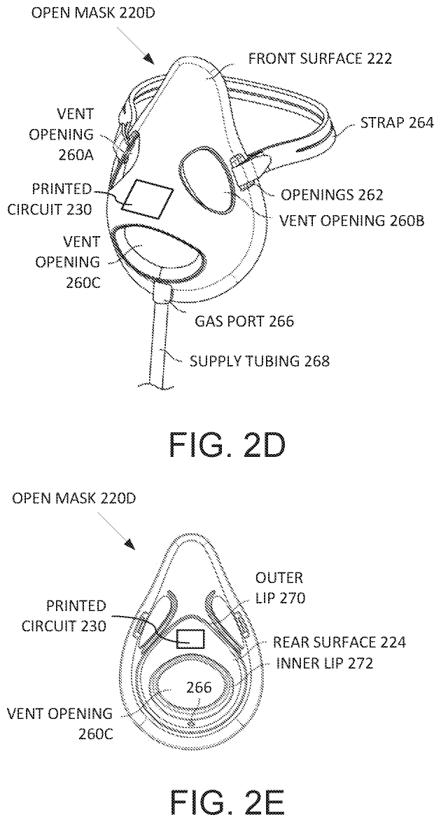

With a high-level overview of system 100 now in place, it may be instructive to examine an example breathing device 120 in conjunction with an example indicator circuit 130. FIG. 2A and FIG. 2B depict an example mask 220 using a printed circuit 230 to indicate CO2 levels during patient inhalation and exhalation, according to various aspects of the subject technology. With respect to FIGS. 2A and 2B, mask 220 may correspond to breathing device 120 from FIG. 1, printed circuit 230 may correspond to indicator circuit 130 from FIG. 1, controller 240 may correspond to controller 140 from FIG. 1, CO2 sensors 250 may correspond to sensors 150 from FIG. 1, and OLED array 260 may correspond to indicators 160 from FIG. 1.

As indicated in printed circuit 230, CO2 sensors 250 are utilized for sensing, but any number of other sensors may be substituted or added. For example, temperature, humidity, breath biomarkers, oxygen sensors, and other sensors may be used. Similarly, while OLED array 260 is utilized for visible indication, other devices can be utilized such as a phosphorescent display, a luminescent element, a LED array, or another device. A flexible indicator, such as a flexible OLED array, may be preferable to facilitate installation of printed circuit 230 on various breathing devices. While a small square shape is illustrated for OLED array 260, any size and shape may be utilized. For example, if mask 220 was instead a nasal cannula, then OLED array 260 may be arranged to align along the patient's nose cavity. Portions of mask 220 including OLED array 260 may also be partially or fully transparent to provide an unobstructed view of a patient wearing mask 220.

Printed circuit 230 may include several components that are not specifically shown in FIG. 2A-2B. For example, printed circuit 230 may include a communications device such as communications device 170 from FIG. 1, a power source such as a printed battery, and conductive printed traces or conductive inks to provide electrical connections between components of printed circuit 230. Further, mask 220 may be fitted on a person's face, such as patient 110 from FIG. 1.

The components of printed circuit 230 may be attached to one or more surfaces of mask 220, including front surface 222 and/or rear surface 224. For example, depending on the permeability and transparency of the materials used for mask 220, a component may be preferably attached to one of front surface 222 or rear surface 224. For example, if mask 220 is largely opaque, then the OLED array 260 may be preferably attached to front surface 222 for visibility. Similarly, if mask 220 has low permeability, then CO2 sensors 250 may be preferably attached to rear surface 224 for optimal detection of the patient's breath. In some embodiments, printed circuit 230 may be a multilayer printed circuit.

Printed circuit 230 may be integrally manufactured with mask 220, for example by direct printing, deposition, insertion into a mold, or other methods. Alternatively, printed circuit 230 may be printed on a separate substrate that is attached to mask 220 by an attachment device. For example, the attachment device may include adhesives, connectors, fasteners, magnets, clasps, straps, buckles, or other similar devices.

As shown in FIG. 2A, controller 240 may direct OLED array 260 to light up, as indicated by cross shading, when sufficient CO2 is detected by CO2 sensors 250, for example by satisfying a high CO2 threshold value. A person wearing mask 220, for example patient 110 of FIG. 1, may generate exhalation wave 212 from normal breathing. The CO2 present in exhalation wave 212 may then be detected via CO2 sensors 250. For example, CO2 sensors 250 may include detection elements that undergo morphological, chemical, or electrical changes proportionally in response to changing CO2 levels, which are then detectable and interpretable by controller 240.

The detection elements of CO2 sensors 250 may include biosensor technology such as cantilever beam based Bio-Micro-Electro-Mechanical Systems (Bio-MEMS), which can provide a detectable voltage proportional to detected CO2 levels. Detection elements may also include printable nanoparticle inks that exhibit detectable electrical changes, for example electrical resistance that changes in proportion to CO2 levels. Detection elements may also include CO2 responsive polymers that undergo detectable morphological changes, such as changing from low to high viscosity fluid and vice versa, or polymer brushes changing between a collapsed and an extended physical state depending on the presence or absence of CO2. Other detection element options include Janus particles and hydrogel sensors. The above recited detection elements may advantageously maintain detection performance over time by avoiding CO2 saturation. In alternative embodiments, other gases or elements may be detected, such as oxygen (O2).

While color changing chemical elements can still be used, for example by using photodiodes to detect color changes in the chemical elements, this may be less preferable due to the previously described special packaging requirements and CO2 saturation issues. Thus, in some preferred embodiments, the detection elements may omit or exclude any chemically reactive color changing materials.

Turning to FIG. 2B, the CO2 sensors 250 may indicate a lower level of CO2 compared to FIG. 2A. For example, the patient may inhale, causing inhalation wave 214 to be drawn towards mask 220. Thus, the CO2 from the prior exhalation wave 212 may dissipate as it is displaced by atmospheric or O2 enriched air. Either way, controller 240 may detect the lower level of CO2 as satisfying a low CO2 threshold value, and in response drive OLED array 260 to become black or transparent, as indicated by the lack of cross shading.

While the above illustrated example uses two thresholds, other embodiments may use several thresholds or ranges. For example, CO2 levels may be detected as falling within five (5) different ranges: very high, high, medium, low, or very low, wherein each range may be calibrated to correspond to a predetermined breathing state such as heavy/moderate/light exhalation, neutral, or inhalation. A detected CO2 level may be indicated by driving OLED array 260 with a specific color associated with the range the CO2 level falls into; for example, bright green for very high CO2 or heavy exhalation, light green for high CO2 or moderate exhalation, yellow for medium CO2 or weak exhalation, light red for low CO2 or neutral, and dark red for very low CO2 or inhalation.

Alternatively or additionally, one or more visual parameters such as the color, hue, area, shape, intensity or brightness of OLED array 260 may be changed proportionally according to the detected level of CO2, for example by becoming brighter with higher detected CO2 levels and dimmer with lower detected CO2 levels. Additionally or alternatively, OLED array 260 may change colors proportionally according to a color gradient, such as increasingly green for high CO2 and increasingly red for low CO2. While this example focuses solely on CO2, if other sensors are present, other sensor data such as temperature, humidity, and various breath biomarkers including O2 levels may also be used to drive the visual parameters of OLED array 260.

Exemplary masks for use with the enclosed disclosure are illustrated in FIG. 2C-2E. Printed circuit 230 may be printed and laminated on a mask interior and/or exterior, disposed around at least one of the mouth or nose. For example, FIG. 2C depicts alternative breathing devices using a printed electronics breath indicator to indicate CO2 levels during patient inhalation and exhalation, according to various aspects of the subject technology. As shown in FIG. 2C, nasal cannula 220A may include printed circuit 230 for each nasal passage, whereas venturi mask 220B and surgery mask 220C may each integrate printed circuit 230 on an inner and/or outer wall and in proximity to the nose and mouth of the patient. Thus, any type of breathing device can utilize printed circuit 230 when an indication of breath is desirable. Further, as illustrated with nasal cannula 220A, multiple positions of printed circuit 230 may be utilized when separate coverage of different breathing areas is desired. When multiple positions of printed circuit 230 are utilized, circuitry may be shared between positions, if feasible.

FIG. 2D depicts a perspective view of open mask 220D using a printed electronics breath indicator to indicate CO2 levels during patient inhalation and exhalation, according to various aspects of the subject technology. As shown in FIG. 2D, open mask 220D includes several openings, or vent opening 260A, vent opening 260B, and vent opening 260C. Strap 264 may be secured to openings 262, e.g. by a hook and loop fastener, to attach open mask 220D to a patient. Gas port 266 may be connected to supply tubing 268 to receive oxygen, for example. Printed circuit 230 may be disposed on front surface 222 to provide a highly visible indication of breath. Alternatively or additionally, printed circuit 230 may also be disposed on rear surface 224, as shown in FIG. 2E, for optimal detection of the patient's breath.

FIG. 2E depicts a rear view of open mask 220D of FIG. 2D, according to various aspects of the subject technology. As shown in FIG. 2E, printed circuit 230 may be disposed above vent opening 260C on rear surface 224, positioned between inner lip 272 and outer lip 270. In some implementations, printed circuit 230 may be a multi-layered film that is disposed on both front surface 222 and rear surface 224.

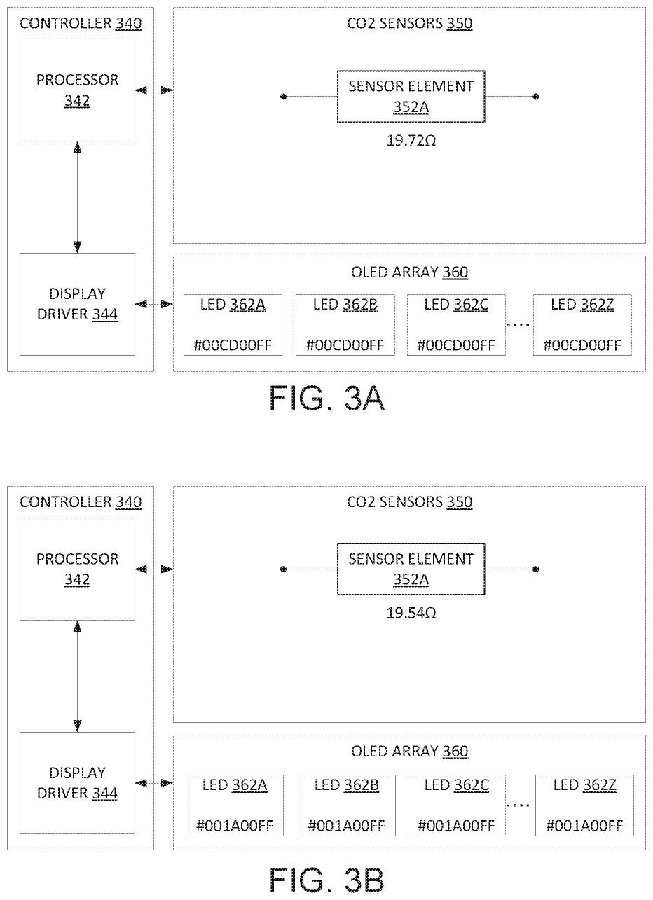

FIG. 3A and FIG. 3B depict a close-up view of example components of printed circuit 230 during patient inhalation and exhalation, according to various aspects of the subject technology. FIG. 3A and FIG. 3B include controller 340, CO2 sensors 350, and OLED array 360, which may correspond to like numbered elements from FIG. 2A and FIG. 2B. Controller 340 includes processor 342 and display driver 344. CO2 sensors 350 includes sensor element 352A. OLED array 360 includes LED 362A, LED 362B, LED 362C, and LED 362Z.

FIG. 3A may correspond to a period when the patient is exhaling, which provides a high CO2 level. In some implementations, sensor element 352A may correspond to a printed nanoparticle ink exhibiting a property of electrical resistance that is proportional to CO2 in the atmosphere. Processor 342 may read the state of sensor elements 352A to determine that the reading of 19.72 ohms corresponds to a high level of CO2, and may correspondingly drive display driver 344 to set LEDs 362A-362Z of OLED array 360 to an opaque green of approximately 80% intensity, or #00CD00FF (e.g., 32-bit RGBA format, or red green blue alpha).

Similarly, FIG. 3B may correspond to a period when the patient is inhaling, which provides a low CO2 level. Accordingly, processor 342 may determine that the reading of 19.54 ohms from sensor element 352 corresponds to a low CO2 level, and may correspondingly drive display driver 344 to set LEDs 362A-362Z of OLED array 360 to an opaque green of approximately 10% intensity, or #001A00FF.

However, as discussed above, threshold values may also be used instead of proportional intensity. For example, a high CO2 level threshold may be defined as exceeding 19.70 ohms and a low CO2 level threshold may be defined as less than 19.56 ohms. In this case, LED 362A-362Z in FIG. 3A may be driven to #00FF00FF, or max brightness green, since 19.72 exceeds 19.70, whereas LED 362A-362Z in FIG. 3B may be driven to #00000000, or transparent, since 19.54 is less than 19.56. Of course, these color values and thresholds are merely illustrative, and any values and thresholds may be used according to use-case requirements, material properties, and calibration results.

In some implementations, thresholds and ranges may change over time based on data recorded for a particular patient, enabling the indicator circuit to provide a customized response for each individual patient. Additionally or alternatively, the thresholds and ranges may change over time based on aggregated data for patient groups or populations. Machine learning techniques may also be used to adjust thresholds and ranges.

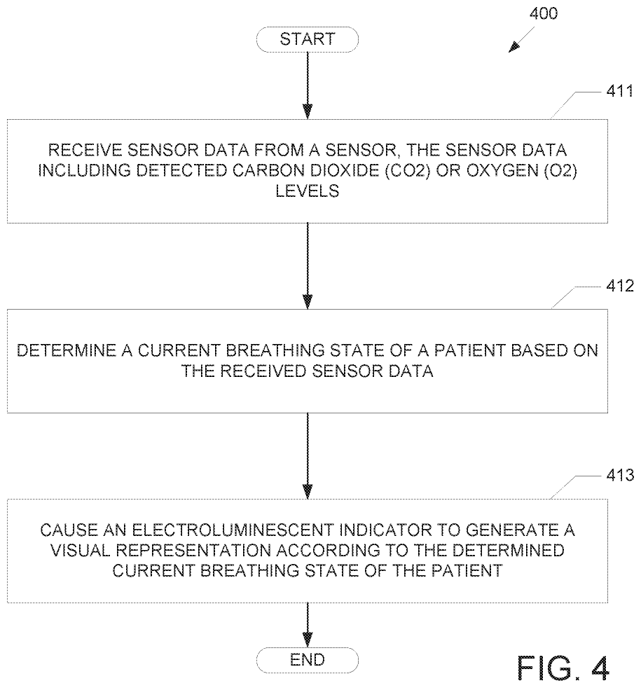

FIG. 4 depicts an example process 400 for a printed electronics breath indicator circuit to provide an indication of breath, according to various aspects of the subject technology. For explanatory purposes, the various blocks of example process 400 are described herein with reference to FIGS. 1-3B, and the components and/or processes described herein. The one or more of the blocks of process 400 may be implemented, for example, by a computing device, including a processor and other components utilized by the device. In some implementations, one or more of the blocks may be implemented apart from other blocks, and by one or more different processors or devices. Further for explanatory purposes, the blocks of example process 400 are described as occurring in serial, or linearly. However, multiple blocks of example process 400 may occur in parallel. In addition, the blocks of example process 400 need not be performed in the order shown and/or one or more of the blocks of example process 400 need not be performed.

In the depicted example flow diagram, sensor data is received from a sensor, the sensor data including detected CO2 or O2 levels (411). Referring to FIG. 1, this may correspond to controller 140 receiving sensor data from sensors 150. Using the example shown in FIG. 3A, this may correspond to receiving information from CO2 sensors 350 indicating an electrical resistance of 19.72 ohms, which may then be translated to an atmospheric CO2 percentage level using a calibrated lookup table, formula, or another method.

Controller 140 may continue to determine a current breathing state of patient 110 based on the received sensor data (412). For example, the CO2 percentage level may be determined to fall under a predetermined CO2 range that corresponds to a predetermined patient breathing state, such as heavy exhalation. As discussed above, the collected sensor data may also include other measurements depending on the sensors included in sensors 150.

Controller 140 may continue to cause indicators 160 to generate a visual representation according to the determined current breathing state of patient 110 (413). For example, when the predetermined patient breathing state is determined to be heavy exhalation, then visual parameters of indicators 160 may be set to display an opaque bright green to indicate a high CO2 level. Alternatively, the visual parameters may be set variably or proportionally according to the CO2 level indicated by the determined current breathing state, which may include light intensity/brightness, color, transparency, area, or other parameters. For example, referring to FIG. 3A, data may be transmitted to display driver 344, which in turn drives LEDs 362A-362Z to the chosen colors, intensities, and transparencies according to the determined visual parameters for the visual representation. OLED array 360 is thus variably driven to display the requested visual representation, whether it is opaque green, transparent, or any other representation. The indicators 160 can then be readily perceived and understood by healthcare provider 112 so that any unexpected breathing conditions of patient 110 can be quickly recognized and acted upon.

The blocks of process 400 may be periodically repeated until available power sources are exhausted, or when a user switches a power toggle, when available. Further, the update rate of the periodic repeating may be adjusted to balance power consumption while providing a sufficiently timely and smoothly updated representation of the current breathing state of the patient 110. As a non-limiting example, the update rate may be set to anywhere between 1 to 30 times per second, depending on the specific use case. Additionally, the update rate may be adjusted based on estimated remaining power available.

As discussed above, controller 140 may also optionally transmit the received sensor data to a remote device, such as remote monitoring device 180, via communications device 170. As discussed above, the sensor data may be aggregated in data store 195 for concurrent or future analysis using analysis software 192 or other programs.

In some implementations, the blocks of process 400 may be performed directly by sensors 150, rather than using controller 140. For example, sensors 150 may undergo morphological or electrical changes in response to changing CO2 or O2 levels. In turn, these changes may directly modulate OLED array 360 or other indicators 160, for example by providing a variable brightness or other visual indication. In this manner, several steps of A/D and D/A conversion may be bypassed, and indicator circuit 130 may be further simplified by omitting controller 140 and communications device 170.

Many aspects of the above-described example process 400, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

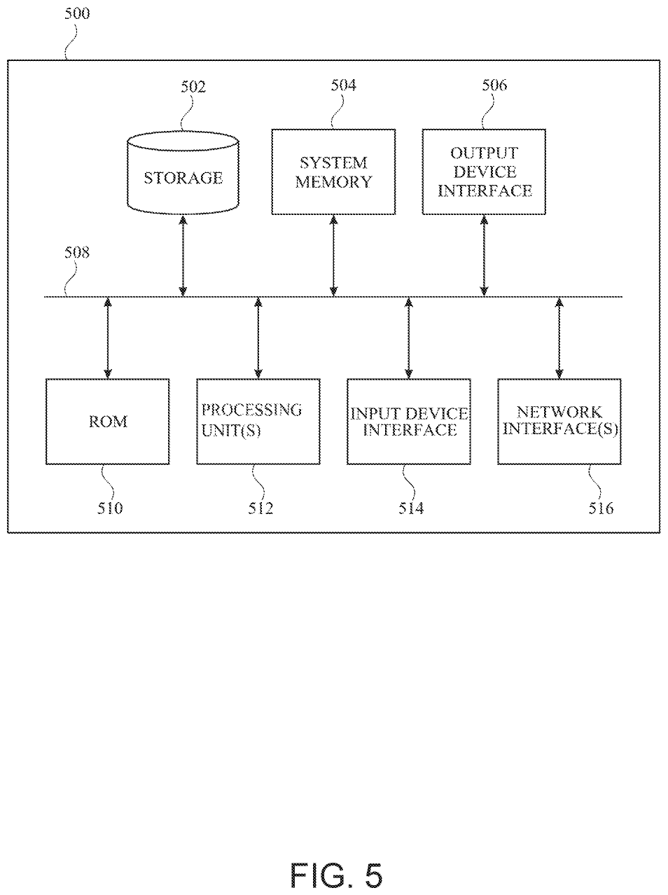

FIG. 5 is a conceptual diagram illustrating an example electronic system 500 for implementation with or within an electronic breath indicator circuit, according to various aspects of the subject technology. Electronic system 500 may be a computing device for execution of software associated with one or more portions or steps of process 400, or components and processes provided by FIGS. 1-4. Electronic system 500 may be representative, in combination with the disclosure regarding FIGS. 1-4, of the indicator circuit 130, 230 and/or the remote monitoring device 180 described above. In this regard, electronic system 500 may be a microcomputer, personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and processes previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such a random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An electronics breath indicator circuit for indicating a breathing state of a patient, comprising: a flexible substrate; an electroluminescent indicator printed on or affixed to the substrate and configured to generate a visual representation according to a received input signal; a sensor printed on or affixed to the substrate; and a processor printed on or affixed to the substrate, wherein the processor is configured to: receive sensor data from the sensor, the sensor data including detected carbon dioxide (CO2) or oxygen (O2) levels; determine, responsive to receiving the sensor data, a current breathing state of the patient based on the received sensor data; and cause, responsive to determining the current breathing state, the electroluminescent indicator to generate the visual representation according to the determined current breathing state of the patient.

Clause 2. The electronics breath indicator circuit of Clause 1, wherein the visual representation comprises a variable visual display of one or more visual parameters that are proportional to the detected CO2 or O2 levels.

Clause 3. The electronics breath indicator circuit of Clause 2, wherein the one or more visual parameters include at least one of brightness, color, and transparency.

Clause 4. The electronics breath indicator circuit of Clause 1, wherein determining the current breathing state is based on the received sensor data satisfying a threshold value or range for a predetermined breathing state.

Clause 5. The electronics breath indicator circuit of Clause 1, wherein the electroluminescent indicator comprises a flexible organic light-emitting diode (OLED) array.

Clause 6. The electronics breath indicator circuit of Clause 1, wherein the sensor comprises at least one of cantilever beams, CO2 or O2 responsive polymers, nanoparticle inks, Janus particles, and hydrogel sensors.

Clause 7. The electronics breath indicator circuit of Clause 1, wherein the sensor excludes any chemically reactive color changing materials.

Clause 8. The electronics breath indicator circuit of Clause 1, further comprising a wireless communication device, and wherein the processor is further configured to transmit the received sensor data to a remote device using the communication device.

Clause 9. The electronics breath indicator circuit of Clause 1, further comprising an attachment device that is attachable to a breathing device.

Clause 10. The electronics breath indicator circuit of Clause 1, further comprising one or more energy devices configured to receive power wirelessly from a power source external to the electronics breath indicator circuit, and to power the processor, sensor, and electroluminescent indicator from the wirelessly received power.

Clause 11. The electronics breath indicator circuit of Clause 1, wherein the flexible substrate is selected from a group consisting of a flexible translucent substrate and a flexible transparent substrate.

Clause 12. The electronics breath indicator circuit of Clause 1, wherein the sensor data includes at least one of temperature, humidity, breath biomarkers, pressure level, and pH level.

Clause 13. A method for an electronics breath indicator to indicate a breathing state of a patient, the method comprising: receiving sensor data from a sensor, the sensor data including detected carbon dioxide (CO2) or oxygen (O2) levels; determining a current breathing state of the patient based on the received sensor data, and causing an electroluminescent indicator to generate a visual representation according to the determined current breathing state of the patient; and wherein the method is performed by one or more processors.

Clause 14. The method of Clause 13, wherein the visual representation comprises a variable visual display of one or more visual parameters that are proportional to the detected CO2 or O2 levels, and wherein the one or more visual parameters include at least one of brightness, color, and transparency.

Clause 15. The method of Clause 13, wherein determining the current breathing state of the patient is based on the received sensor data satisfying a threshold value or a range for a predetermined breathing state.

Clause 16. The method of Clause 13, wherein causing the electroluminescent indicator to generate the visual representation comprises driving a display driver to light up a flexible organic light-emitting diode (OLED) array of the electroluminescent indicator.

Clause 17. The method of Clause 13, wherein receiving the sensor data from the sensor comprises receiving from the sensor comprising at least one of cantilever beams, CO2 or O2 responsive polymers, nanoparticle inks, Janus particles, and hydrogel sensors.

Clause 18. A system for indicating a breathing state of a patient, comprising: substrate means; electroluminescent indicator means printed on or affixed to the substrate means; sensor means printed on or affixed to the substrate means; and processing means printed on or affixed to the substrate means, wherein the processing means comprises: receiving means for receiving sensor data from the sensor means, the sensor data including detected carbon dioxide (CO2) or oxygen (O2) levels; determining means for determining a current breathing state of the patient based on the received sensor data; and causation means for causing the electroluminescent indicator means to generate a visual representation according to the determined current breathing state of the patient.

Clause 19. The system of Clause 18, wherein the visual representation comprises a variable visual display of one or more visual parameters of the visual representation that are proportional to the detected CO2 or O2 levels, wherein the one or more visual parameters include at least one of brightness, color, and transparency.

Clause 20. The system of Clause 18, wherein the processing means further comprises a communications means for transmitting the received sensor data wirelessly to a remote device.

Clause 21. An electronics breath indicator circuit for indicating a breathing state of a patient, comprising: a flexible substrate; an electroluminescent indicator printed on or affixed to the substrate and configured to generate a visual representation according to a received input signal; and a sensor printed on or affixed to the substrate, wherein the sensor is configured to: provide one or more morphological or electrical changes in response to changing carbon dioxide (CO2) or oxygen (O2) levels; and modulate the electroluminescent indicator according to the one or more morphological or electrical changes to provide a visual representation of a current breathing state of the patient.

Clause 22. A method for an electronics breath indicator to indicate a breathing state of a patient, the method comprising: providing, by a sensor printed on or affixed to a flexible substrate, one or more morphological or electrical changes in response to changing carbon dioxide (CO2) or oxygen (O2) levels; and modulate, by the sensor, an electroluminescent indicator printed on or affixed to the flexible substrate, the modulating according to the one or more morphological or electrical changes to provide a visual representation of a current breathing state of the patient.

Further Consideration

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit this disclosure.

The term website, as used herein, may include any aspect of a website, including one or more web pages, one or more servers used to host or store web related content, etc. Accordingly, the term website may be used interchangeably with the terms web page and server. The predicate words “configured to,” “operable to,” and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. An electronics breath indicator circuit for indicating a breathing state of a patient, comprising:
a flexible substrate;
an electroluminescent indicator printed on or affixed to the substrate and configured to generate a visual representation according to a received input signal;
a sensor printed on or affixed to the substrate; and
a processor printed on or affixed to the substrate, wherein the processor is configured to:
receive sensor data from the sensor, the sensor data including detected carbon dioxide (CO2) or oxygen (O2) levels;
determine, responsive to receiving the sensor data, a current breathing state of the patient based on the received sensor data; and
cause, responsive to determining the current breathing state, the electroluminescent indicator to generate the visual representation according to the determined current breathing state of the patient.

2. The electronics breath indicator circuit of claim 1, wherein the visual representation comprises a variable visual display of one or more visual parameters that are proportional to the detected CO2 or O2 levels.

3. The electronics breath indicator circuit of claim 2, wherein the one or more visual parameters include at least one of brightness, color, and transparency.

4. The electronics breath indicator circuit of claim 1, wherein determining the current breathing state is based on the received sensor data satisfying a threshold value or range for a predetermined breathing state.

5. The electronics breath indicator circuit of claim 1, wherein the electroluminescent indicator comprises a flexible organic light-emitting diode (OLED) array.

6. The electronics breath indicator circuit of claim 1, wherein the sensor comprises at least one of cantilever beams, CO2 or O2 responsive polymers, nanoparticle inks, Janus particles, and hydrogel sensors.

7. The electronics breath indicator circuit of claim 1, wherein the sensor excludes any chemically reactive color changing materials.

8. The electronics breath indicator circuit of claim 1, further comprising a wireless communication device, and wherein the processor is further configured to transmit the received sensor data to a remote device using the communication device.

9. The electronics breath indicator circuit of claim 1, further comprising an attachment device that is attachable to a breathing device.

10. The electronics breath indicator circuit of claim 1, further comprising one or more energy devices configured to receive power wirelessly from a power source external to the electronics breath indicator circuit, and to power the processor, sensor, and electroluminescent indicator from the wirelessly received power.

11. The electronics breath indicator circuit of claim 1, wherein the flexible substrate is selected from a group consisting of a flexible translucent substrate and a flexible transparent substrate.

12. The electronics breath indicator circuit of claim 1, wherein the sensor data includes at least one of temperature, humidity, breath biomarkers, pressure level, and pH level.

13. A method for an electronics breath indicator to indicate a breathing state of a patient, the method comprising:
receiving sensor data from a sensor, the sensor data including detected carbon dioxide (CO2) or oxygen (O2) levels;
determining a current breathing state of the patient based on the received sensor data; and
causing an electroluminescent indicator to generate a visual representation according to the determined current breathing state of the patient; and
wherein the method is performed by one or more processors.

14. The method of claim 13, wherein the visual representation comprises a variable visual display of one or more visual parameters that are proportional to the detected CO2 or O2 levels, and wherein the one or more visual parameters include at least one of brightness, color, and transparency.

15. The method of claim 13, wherein determining the current breathing state of the patient is based on the received sensor data satisfying a threshold value or a range for a predetermined breathing state.

16. The method of claim 13, wherein causing the electroluminescent indicator to generate the visual representation comprises driving a display driver to light up a flexible organic light-emitting diode (OLED) array of the electroluminescent indicator.

17. The method of claim 13, wherein receiving the sensor data from the sensor comprises receiving from the sensor comprising at least one of cantilever beams, CO2 or O2 responsive polymers, nanoparticle inks, Janus particles, and hydrogel sensors.

18. A system for indicating a breathing state of a patient, comprising:
substrate means;
electroluminescent indicator means printed on or affixed to the substrate means;
sensor means printed on or affixed to the substrate means; and
processing means printed on or affixed to the substrate means, wherein the processing means comprises:
receiving means for receiving sensor data from the sensor means, the sensor data including detected carbon dioxide (CO2) or oxygen (O2) levels;
determining means for determining a current breathing state of the patient based on the received sensor data; and
causation means for causing the electroluminescent indicator means to generate a visual representation according to the determined current breathing state of the patient.

19. The system of claim 18, wherein the visual representation comprises a variable visual display of one or more visual parameters of the visual representation that are proportional to the detected CO2 or O2 levels, wherein the one or more visual parameters include at least one of brightness, color, and transparency.

20. The system of claim 18, wherein the processing means further comprises a communications means for transmitting the received sensor data wirelessly to a remote device.

21. An electronics breath indicator circuit for indicating a breathing state of a patient, comprising:
a flexible substrate;
an electroluminescent indicator printed on or affixed to the substrate and configured to generate a visual representation according to a received input signal; and
a sensor printed on or affixed to the substrate, wherein the sensor is configured to:
provide one or more morphological or electrical changes in response to changing carbon dioxide (CO2) or oxygen (O2) levels; and
modulate the electroluminescent indicator according to the one or more morphological or electrical changes to provide a visual representation of a current breathing state of the patient.

22. A method for an electronics breath indicator to indicate a breathing state of a patient, the method comprising:
providing, by a sensor printed on or affixed to a flexible substrate, one or more morphological or electrical changes in response to changing carbon dioxide (CO2) or oxygen (O2) levels; and
modulate, by the sensor, an electroluminescent indicator printed on or affixed to the flexible substrate, the modulating according to the one or more morphological or electrical changes to provide a visual representation of a current breathing state of the patient.
